(19) United States
(12) Patent Application Publication
(10) Pub. No.: US 2024/0360515 A1
(43) Pub. Date: Oct. 31, 2024

(54) MONITORING CIRCULATING TUMOR DNA TO IMPROVE SUBCLONE PENETRATION OF FOLLOW-UP NEOANTIGEN CANCER VACCINES

(71) Applicant: Amazon Technologies, Inc., Reno, NV (US)

(72) Inventors: Layne Christopher PRICE, Seattle, WA (US); Jasleen Kaur GREWAL, Vancouver (CA); David HECKERMAN, Bellevue, WA (US); Frank Wilhelm SCHMITZ, Seattle, WA (US); Melanie WALKER, Seattle, WA (US)

(21) Appl. No.: 18/029,044

(22) PCT Filed: Mar. 16, 2023
(86) PCT No.: PCT/US23/64512

§ 371 Date: Mar. 28, 2023

Related U.S. Application Data
(60) Provisional application No. 63/320,563, filed on Mar. 16, 2022.

(51) Int. Cl. C12Q 1/6886; C12Q 1/6806; C12Q 1/6874

(52) U.S. Cl. CPC C12Q 1/6886; C12Q 1/6874; C12Q 1/6806; C12Q 2600/156

(57) ABSTRACT

Disclosed herein are methods for evaluating the efficacy of an immunogenic composition by monitoring circulating tumor DNA. The disclosure also relates to methods for treating a subject having cancer.

### 1. CROSS-REFERENCE TO RELATED APPLICATIONS

This application claims the benefit of U.S. Provisional Patent Application 63/320,563, filed Mar. 16, 2022, the entire contents of which are incorporated herein by reference.

### 2. BACKGROUND

Cancer is a leading cause of death worldwide accounting for 1 in 4 of all deaths. Siegel et al., CA: A Cancer Journal for Clinicians, 68:7-30 (2018). There were 18.1 million new cancer cases and 9.6 million cancer-related deaths in 2018. Bray et al., CA: A Cancer Journal for Clinicians, 68(6):394-424. There are a number of existing standard of care cancer therapies, including ablation techniques (e.g., surgical procedures and radiation) and chemical techniques (e.g., chemotherapeutic agents). Unfortunately, such therapies are frequently associated with serious risk, toxic side effects, and extremely high costs, as well as uncertain efficacy.

Cancer immunotherapy (e.g., cancer vaccine) has emerged as a promising cancer treatment modality. The goal of cancer immunotherapy is to harness the immune system for selective destruction of cancer while leaving normal tissues unharmed. Traditional cancer vaccines typically target tumor-associated antigens. Tumor-associated antigens are typically present in normal tissues, but overexpressed in cancer. However, because these antigens are often present in normal tissues immune tolerance can prevent immune activation. Several clinical trials targeting tumor-associated antigens have failed to demonstrate a durable beneficial effect compared to standard of care treatment. Li et al., Ann Oncol., 28 (Suppl 12): xii11-xii17 (2017).

One of the hallmarks of cancer is the accumulation of somatic alterations, acquired during a cell's life cycle and development. Shen, J. Mol Cell Biol. 3(1):1-3 (2011). Throughout cancer progression, some of these variants may dictate response to therapy. Dancey et al., Cell, 148(3):409-420 (2012).

As such, neoantigens represent an attractive target for cancer immunotherapies. Neoantigens are non-autologous proteins with individual specificity. Neoantigens are derived from random somatic mutations (e.g., somatic single nucleotide variants and small insertions and deletions (indels)) in the tumor cell genome and are not expressed on the surface of normal cells. Id. Because neoantigens are expressed exclusively on tumor cells, and thus do not induce central immune tolerance, cancer vaccines targeting cancer neoantigens have potential advantages, including decreased central immune tolerance and an improved safety profile. Id

The mutational landscape of cancer is complex and tumor mutations are generally unique to each individual subject. Most somatic mutations detected by sequencing do not result in effective neoantigens. Only a small percentage of mutations in the tumor DNA, or a tumor cell, are transcribed, translated, and processed into a tumor-specific neoantigen with sufficient accuracy to design a vaccine that is likely to be effective. Further, not all neoantigens are immunogenic.

The reliable detection of neoantigens (i.e., mutations) in cancer genomes is important for developing effective therapies as well as guiding treatment choices for cancer, such as immunogenic compositions. Identification of somatic mutations is challenged by the heterogeneous composition of tumors. Evaluating heterogeneity to guide choice and sequence of therapy could be achieved by tumor biopsies, but this is impractical due to the associated risk of complications and costs. Alternatively, circulating tumor-derived DNA (ctDNA) can be used to monitor cancer dynamics noninvasively. Circulating tumor derived DNA is DNA originating from tumor cells (e.g., from primary tumors, micrometastases, and overt metastases) that is released into the circulation. Fiala et al., (2018), The Journal of Applied Laboratory Medicine, 3(2):300-313. However, the sensitivity of circulating DNA analysis is limited by extremely low amounts of tumor DNA concentrations in the blood and by the methods of detection. Id. Currently, circulating tumor DNA is unlikely to perform at the high level of sensitivity and specificity required to apply clinically. Id.

Accordingly, there is a significant unmet need for an integrated method that characterizes tumor genomic material to identify neoantigens and selects which neoantigens are likely to be suitable for effective immunogenic compositions.

### 3. SUMMARY

The disclosure relates to methods for evaluating the efficacy of an immunogenic composition. The methods comprise sequencing the circulating tumor DNA from a biological sample of a subject to generate circulating tumor DNA sequence data. Next, the circulating tumor DNA sequence data is analyzed to generate a numerical probability score of the prevalence of one or more tumor-specific neoantigens in the circulating tumor DNA. A higher numerical probability score relative to a low numerical probability score indicates that the one or more tumor-specific neoantigens is present in the circulating tumor DNA and that the one or more tumor-specific neoantigens has a lower prevalence in a tumor of the subject relative to before administration of the immunogenic composition. A lower numerical probability score relative to a higher numerical probability score indicates that the one or more tumor specific neoantigens is present in a low amount or not present in the circulating tumor DNA and that the one or more tumor-specific neoantigens has a higher prevalence in the tumor of the subject relative to before administration of the immunogenic composition. The method can further comprise generating a second immunogenic composition.

The disclosure also relates to methods for treating cancer. The methods comprise administering to a subject in need thereof a first immunogenic composition. Next, the circulating tumor DNA from a biological sample from a subject is sequenced to generate circulating tumor DNA sequence data. The circulating tumor DNA sequence data is then analyzed to generate a numerical probability score of the prevalence of one or more tumor-specific neoantigens in the circulating tumor DNA. A higher numerical probability score indicates that the one or more tumor-specific neoantigens is present in the circulating tumor DNA and that the one or more tumor-specific neoantigens has a lower prevalence in a tumor of the subject relative to before administration of the first immunogenic composition. A lower numerical probability score indicates that the one or more tumor specific neoantigens is present in a low amount or not present in the circulating tumor DNA and that the one or more tumor-specific neoantigens has a higher prevalence in the tumor of the subject relative to before administration of the first immunogenic composition. Lastly, the method comprises generating a second immunogenic composition. The methods can further comprise clustering the one or more tumor-specific neoantigens identified in the circulating tumor DNA to identify one or more tumor-subclones.

The subject was previously administered or will be prospectively administered an immunogenic composition. The immunogenic composition can be a tumor-specific neoantigen immunogenic composition or a tumor-associated antigen-based immunogenic composition. The first immunogenic composition and the second immunogenic composition can be a tumor-specific neoantigen immunogenic composition or a tumor-associated antigen-based immunogenic composition.

The second immunogenic composition can comprise one or more tumor-specific neoantigens that have a lower numerical probability score relative to a higher numerical probability score. The second immunogenic composition can comprises one or more tumor-specific neoantigens not present in the circulating tumor DNA. The second immunogenic composition can comprise one or more tumor-specific neoantigens that are present in the circulating tumor DNA.

In some embodiments, one or more tumor-specific neoantigens associated with the identified one or more tumor-subclones are not included in the second immunogenic composition. In some embodiments, one or more tumor-specific neoantigens associated with the identified one or more tumor-subclones are included in the second immunogenic composition. In some embodiments, tumor-specific neoantigens identified in the circulating tumor DNA and tumor-specific neoantigens identified as being associated with one or more tumor-subclones of the tumor-specific neoantigens identified in the circulating tumor DNA are not included in the second immunogenic composition.

Absence of the one or more tumor-specific neoantigens in the circulating tumor DNA indicates that the tumor-specific neoantigens are prevalent in a tumor of the subject.

The disclosure also relates to methods for determining whether a subject has cancer relapse. The methods comprise sequencing the circulating tumor DNA from a biological sample of the subject to generate circulating tumor DNA sequence data, and analyzing the circulating tumor DNA sequence data to generate a numerical probability score of the prevalence of one or more tumor-specific neoantigens in the circulating tumor DNA. A higher numerical probability score relative to a low numerical probability score indicates that the one or more tumor-specific neoantigens is present in the circulating tumor DNA and the subject has cancer relapse. A lower numerical probability score relative to a higher numerical probability score indicates that the one or more tumor specific antigens is present in a low amount or not present in the circulating tumor DNA and that the subject does not have cancer re-lapse. The method can further comprise generating an immunogenic composition. The subject was previously administered or will be prospectively administered an immunogenic composition. The method can further comprise clustering the one or more tumor-specific neoantigens identified in the circulating tumor DNA to identify one or more tumor-subclones. The immunogenic composition can be a tumor-specific neoantigen immunogenic composition or a tumor-associated antigen-based immunogenic composition. The immunogenic composition can comprise one or more tumor-specific neoantigens that have a higher numerical probability score relative to a lower numerical probability score. The immunogenic composition can comprise one or more tumor-specific neoantigens present in the circulating tumor DNA. The presence of the one or more tumor-specific neoantigens in the circulating tumor DNA can indicate that the subject has cancer relapse.

The one or more tumor-specific neoantigens associated with the identified one or more tumor-subclones can be included in the immunogenic composition. The tumor-specific neoantigens identified in the circulating tumor DNA and tumor-specific neoantigens identified as being associated with one or more tumor-subclones of the tumor-specific neoantigens identified in the circulating tumor DNA can be included in the immunogenic composition.

The circulating tumor DNA can be obtained from a whole blood sample. For example, circulating tumor DNA is obtained from a blood sample, a serum sample, a plasma sample, a lymphatic fluid sample, a urine sample, or a cerebrospinal fluid sample. The circulating tumor DNA can be obtained from tumor draining veins (e.g., immediately downstream or locoregional). The circulating tumor DNA can be obtained from lymph downstream (e.g., immediately downstream) from a tumor.

The circulating tumor DNA can be sequenced using whole exome sequencing, whole genome sequencing, targeted sequencing, polymerase chain reaction (PCR), or other sequencing methods.

The circulating tumor DNA can be obtained from the subject at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the immunogenic composition or the first immunogenic composition. The circulating tumor DNA can be obtained from the subject at least about 1 month, about 2 months, about 3 months, about 4 months, about 5 months, about 6 months, about 7 months, about 8 months, about 9 months, about 10 months, about 11 months, or about 12 months after administering the immunogenic composition or the first immunogenic composition. The circulating tumor DNA can be obtained from the subject at least about 1 years, about 2 years, about 3 years, about 4 years, about 5 years, about 6 years, about 7 years, about 8 years, about 9 years, or about 10 years, after administering the immunogenic composition or the first immunogenic composition.

The subject may have cancer. For example, the subject may have been diagnosed with cancer. The subject can have melanoma, breast cancer, sarcomas, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, colon cancer, urothelial cancer, or lung cancer.

### 4. BRIEF DESCRIPTION OF THE DRAWINGS

FIG. 1 is a schematic depicting the approach for selecting one or more tumor-specific neoantigens.

FIG. 2 is schematic flow diagram depicting the bioinformatics analysis of next generation sequencing data (input and output).

FIG. 3 is a flow diagram of the module for clonality deconvolution.

### 5. DETAILED DESCRIPTION

The disclosure relates to a novel approach that uses circulating tumor DNA information to derive estimates on tumor-specific neoantigen cellular prevalence (e.g. what fraction of the tumor cells that the tumor-specific neoantigen is present in). This information can be used to tailor the treatment regimen for a subject having cancer. One or more tumor-specific neoantigens in an immunogenic composition (e.g., personalized cancer vaccine) can be selected based on the information derived from the circulating tumor DNA. Without wishing to be bound by theory, it is believed that tumor-specific neoantigens that are less dominant in the circulating tumor DNA will be biased toward tumor-specific neoantigens that have not elicited a robust immune response. These tumor-specific mutations are likely evading the immune system. Without wishing to be bound by theory, tumor-specific neoantigens that are more dominant in the circulating tumor DNA will be biased toward tumor-specific neoantigens that have elicited a robust immune response. These tumor-specific mutations are responding to either the subject's immune response or a previously administered immunogenic composition.

Generally, tumor-specific neoantigens that appear in the circulating tumor DNA are likely to have a lower cellular prevalence in the tumor. Tumor-specific neoantigens that do not appear in the circulating tumor DNA are likely to have a higher cellular presence in the tumor. For example, tumor-specific neoantigens not present in the circulating tumor DNA have not likely been killed to any significant degree. The circulating tumor DNA information can also be used to derive a tumor sub-clone profile. A tumor sub-clone profile is an estimate for which of the tumor-specific neoantigens occur together in the same population of cancer cells. The tumor sub-clone profile can be used to inform selection of a tumor-specific neoantigen for an immunogenic composition. For example, a tumor sub-clone that is prevalent based on the presence of a tumor-specific neoantigen in the circulating tumor DNA can be excluded from an immunogenic composition.

The approach begins with sequencing circulating tumor DNA from a biological sample of a subject. The subject may have been previously administered an immunogenic composition. Alternatively, the subject will be administered an immunogenic composition. The circulating tumor DNA is sequenced to obtain sequence data. Next, the circulating tumor DNA sequence data is analyzed to generate a numerical probability score of the prevalence of one or more tumor-specific neoantigens in the circulating tumor DNA. A higher numerical probability score relative to a low numerical probability score indicates that one or more tumor-specific neoantigens is present in the circulating tumor DNA and that the one or more tumor-specific neoantigens has a lower prevalence in a tumor of the subject relative to before administration of the immunogenic composition. A lower numerical probability score relative to a higher numerical probability score indicates that the one or more tumor specific neoantigens is present in a low amount or not present in the circulating tumor DNA and that the one or more tumor-specific neoantigens has a higher prevalence in the tumor of the subject relative to before administration of the immunogenic composition. The method uses high-throughput sequencing technology and machine learning platforms to determine the prevalence of one or more tumor-specific neoantigens in the circulating tumor DNA.

The disclosure further relates to methods for treating cancer. The methods disclosed herein can comprise generating an immunogenic composition based on the numerical probability score of the prevalence of one or more tumor-specific neoantigens in the circulating tumor DNA.

Additional description of the methods and guidance for the practice of the methods are provided herein.

### A. Circulating Tumor DNA

Disclosed herein are methods for inferring the prevalence of tumor-specific neoantigens in a tumor (i.e., what fraction of the tumor cells the tumor-specific neoantigen appears in). The methods disclosed herein comprise sequencing circulating tumor DNA from a biological sample from a subject. The circulating tumor DNA is sequenced to obtain sequence data. The sequence data comprises sequence reads of a plurality of polynucleotides from the subject. The circulating tumor DNA is analyzed to generate a numerical probability score of the prevalence of one or more tumor-specific neoantigens in the circulating tumor DNA.

A higher numerical probability score relative to a low numerical probability score indicates that the one or more tumor-specific antigens is present in the circulating tumor DNA and that the one or more tumor-specific neoantigens has a lower prevalence in a tumor of the subject relative to before administration of the immunogenic composition. A lower numerical probability score relative to a higher numerical probability score indicates that the one or more tumor specific antigens is present in a low amount or not present in the circulating tumor DNA, and that the one or more tumor-specific neoantigens has a higher prevalence in the tumor of the subject relative to before administration of the immunogenic composition. A tumor-specific neoantigen with a higher numerical probability score relative to a lower numerical probability score indicates that the tumor-specific neoantigen is present in the circulating tumor DNA and an immune response has been elicited to the tumor-specific neoantigen at the tumor site.

The numerical probability score can be a number between 0 and 1. In embodiments, the numerical probability score can be a number of 0, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, or 1. For example, a tumor-specific neoantigen with a numerical probability score of 1 is likely more prevalent in the circulating tumor DNA and less prevalent in the tumor (e.g., an immune response against the tumor-specific neoantigen has been elicited) in comparison to a tumor-specific neoantigen having a numerical probability score of 0.05. A tumor-specific neoantigen having a numerical probability score of 0.5 is likely less prevalent in the circulating tumor DNA and more prevalent in the tumor (e.g., an immune response against the tumor-specific neoantigen has not been elicited) in comparison to a tumor-specific neoantigen with a numerical probability score of 0.9.

The methods disclosed herein can further comprise generating an immunogenic composition based on the information derived from the circulating tumor DNA. The immunogenic composition can comprise one or more tumor-specific neoantigens that have a lower numerical probability score relative to a higher numerical probability score. The immunogenic composition can comprise one or more tumor-specific neoantigens that have a lower numerical probability score relative to a higher numerical probability score, and one or more tumor-specific neoantigens that have a higher numerical probability score relative to a lower numerical probability score. The immunogenic composition may exclude one or more tumor-specific neoantigens present in the circulating tumor DNA. The immunogenic composition may preferentially include one or more tumor-specific neoantigens not present in the circulating tumor DNA.

The methods disclosed herein can further comprise clustering one or more-tumor specific neoantigens identified in the circulating tumor DNA to identify one or more tumor sub-clones and generate a tumor sub-clone profile. The tumor sub-clone profile can be used to inform selection of a tumor-specific neoantigen for an immunogenic composition. For example, a tumor sub-clone that is prevalent based on the presence of a tumor-specific neoantigen in the circulating tumor DNA may be excluded from an immunogenic composition. For example, a tumor sub-clone that is not associated with one or more tumor-specific neoantigens may preferentially be included in an immunogenic composition. It is preferred that tumor-specific neoantigens that are associated with a tumor sub-clone identified using the circulating tumor DNA are not included in an immunogenic composition. A tumor-specific neoantigen that is associated with a tumor sub-clone that has a lower numerical probability score relative to a higher numerical probability score can be included in an immunogenic composition.

Disclosed herein are methods for determining whether a subject has cancer relapse. The methods disclosed herein comprise sequencing circulating tumor DNA from a biological sample from a subject. The circulating tumor DNA is sequenced to obtain sequence data. The sequence data comprises sequence reads of a plurality of polynucleotides from the subject. The circulating tumor DNA is analyzed to generate a numerical probability score of the prevalence of one or more tumor-specific neoantigens in the circulating tumor DNA. A higher numerical probability score relative to a low numerical probability score indicates that the one or more tumor-specific neoantigens is present in the circulating tumor DNA and the subject has cancer relapse. A lower numerical probability score relative to a higher numerical probability score indicates that the one or more tumor specific antigens is present in a low amount or not present in the circulating tumor DNA and that the subject does not have cancer relapse.

As disclosed herein, the numerical probability score can be a number between 0 and 1. As disclosed herein, the numerical probability score can be a number of 0, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, or 1. For example, a tumor-specific neoantigen with a numerical probability score of 1 is likely more prevalent in the circulating tumor DNA and the subject is more likely to have cancer relapse in comparison to a tumor-specific neoantigen having a numerical probability score of 0.05. A tumor-specific neoantigen having a numerical probability score of 0.5 is likely less prevalent in the circulating tumor DNA and the subject is less likely to have cancer relapse in comparison to a tumor-specific neoantigen with a numerical probability score of 0.9.

The methods disclosed herein can further comprise generating an immunogenic composition based on the information derived from the circulating tumor DNA. The immunogenic composition can comprise one or more tumor-specific neoantigens that have higher numerical probability score relative to a lower numerical probability score. The immunogenic composition may exclude one or more tumor-specific neoantigens not present in the circulating tumor DNA. The immunogenic composition may preferentially include one or more tumor-specific neoantigens present in the circulating tumor DNA.

The methods disclosed herein can further comprise clustering one or more tumor-specific neoantigens identified in the circulating tumor DNA to identify one or more tumor sub-clones and generate a tumor sub-clone profile. The tumor sub-clone profile can be used to inform selection of a tumor-specific neoantigen for an immunogenic composition. For example, a tumor sub-clone that is prevalent based on the presence of a tumor-specific neoantigen in the circulating tumor DNA may be included in an immunogenic composition. For example, a tumor sub-clone that is associated with one or more tumor-specific neoantigens may preferentially be included in an immunogenic composition. It is preferred that tumor-specific neoantigens that are associated with a tumor sub-clone identified using the circulating tumor DNA are included in an immunogenic composition. A tumor-specific neoantigen that is associated with a tumor sub-clone that has a higher numerical probability score relative to a lower numerical probability score can be included in an immunogenic composition.

While the methods disclosed herein preferably measure the presence of one or more tumor-specific neoantigens, the circulating DNA can comprise additional types of mutations, for example, germline mutations. Germline mutations refer to mutations existing in germline DNA of a subject. The methods disclosed herein can be utilized to measure germline mutations.

Exemplary amounts of circulating tumor DNA in a biological sample (e.g., plasma or serum) can range from about 1 femtogram (fg) to about 1000 nanograms (ng), e.g., 1 picogram (pg) to 200 ng, 1 nanogram (ng) to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 fg, 10 fg, 100 fg, 1 pg, 10 pg, 100 pg, 1 ng, 10 ng, 100 ng, 150 ng, or 200 ng of circulating tumor DNA molecules. The method can comprise obtaining 1 fg to 200 ng circulating tumor DNA.

The circulating tumor DNA can have an exemplary size distribution of about 100-500 nucleotides. The circulating tumor DNA can be about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500 nucleotides.

A variety of methods exist for obtaining the genome sequence data described herein. Sequencing methods are well known in the art and include, but are not limited to, whole genome sequencing, whole exome sequencing, targeted sequencing, PCR-based methods, including real-time PCR (RT-PCR), deep sequencing, high-throughput sequencing, or combinations thereof. In some instances, the foregoing techniques and procedures can be performed according to the methods described in e.g., Sambrook et al., Molecular Cloning: A Laboratory Manual 4th ed. (2012) Cold Spring Harbor Laboratory Press, Cold Spring Harbor, NY. See also, Austell et al., Current Protocols in Molecular Biology, ed., Greene Publishing and Wiley-Interscience New York (1992) (with periodic updates).

The genome sequence data can be obtained from whole genome sequencing, whole exome sequencing, targeted sequencing, DNA hybridization methods or combinations thereof. The genome sequencing data can be sequence data derived from high-depth whole genome sequencing data.

The circulating tumor DNA is sequenced to obtain sequence reads. The sequence data comprises sequence reads of a plurality of polynucleotides from the subject. Sequence reads can comprise about 2 to about 5000 nucleotides. For example, about 2, about 3, about 4, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 550, about 600, about 700, about 800, about 900, about 1,000, about 1,100, about 1,200, about 1,300, about 1,400, about 1,500, about 1,600, about 1,700, about 1,800, about 1,900, about 2,000, about 2,100, about 2,200, about 2,300, about 2,400, about 2,500, about 2,600, about 2,700, about 2,800, about 2,900, about 3,000, about 3,100, about 3,200, about 3,300, about 3,400, about 3,500, about 3,600, about 3,700, about 3,800, about 3,900, about 4,000, about 4,100, about 4,200, about 4,300, about 4,400, about 4,500, about 4,600, about 4,700, about 4,800, about 4,900, or about 5,000 nucleotides.

The circulating tumor DNA sequence data can be analyzed using integration of variant reads (INVAR) as described in International Application No.: PCT/EP2019/055610. The circulating tumor DNA sequence data can be analyzed using an objective function outlined in Equation 1:

$$N = {\sum }_{i = 1}^{C} {ξ}_{i} {{⋁}_{α = 1}}^{L} {\tilde{P}}_{i α} {s}_{α} \$ \qquad Eq . 1$$

$

N=i=1 ΣCξiα=1VLP˜iαsα

where ξi is the ith sub-clone's estimated cellular prevalence. P˜iαis the probability that peptide α is in the ith sub-clone, and sa is the individual score of the alpha'th peptide from the machine learning modeling. All of these prevalence and sub-clonality estimates are derived from the combination of biopsy and ctDNA sequencing. The “big-OR” is taken over the best L peptides, with L=20, and the summation is taken over all of the C sub-clones, with C a parameter returned by the sub-clonality estimation algorithms. Typically, 1<C<15.

The circulating tumor DNA sequence data can be analyzed using an objective function outlined in Equation 2:

$$N = {\sum }_{α = 1}^{L} {ξ}_{α} {s}_{α} \qquad Eq . 2$$

N=α=1 ΣL τ α s α

where ξα is the cellular prevalence of the alpha'th mutation and sα is the individual score of the alpha'th peptide from the machine learning modeling. A straight sort of all peptides by the product ξαsα optimizes this function.

The circulating tumor DNA sequence data can be analyzed using a machine learning platform. Exemplary machine learning models that can be suitable include, but are not limited to, a neural network, a Bayesian classifier, a logistic regression, a decision tree, a gradient boosting decision tree, a random forest, a support vector machine, a gradient-boosted tree, a multilayer perceptron, a one-vs-rest, or a Gaussian Naive Bayes.

### B. Methods of Treatment

This disclosure also relates to methods of treating cancer in a subject in need thereof comprising administering a personalized immunogenic composition comprising one or more tumor specific neoantigens selected using the methods described herein.

The methods disclosed herein can comprise administering to a subject in need thereof an immunogenic composition based on the information derived from the circulating tumor DNA. The immunogenic composition can be personalized based on the information derived from the circulating tumor DNA. For example, a tumor-specific neoantigen that has a low numerical probability score may be preferentially included in an immunogenic composition. For example, a tumor-specific neoantigen that has a high numerical probability score may be preferentially excluded from an immunogenic composition. For example, a tumor-specific neoantigen that is associated with a tumor sub-clone identified based on the circulating tumor DNA may be excluded from the immunogenic composition. For example, a tumor-specific neoantigen that is not associated with a tumor sub-clone identified based on the circulating tumor DNA may be preferentially included in an immunogenic composition.

The subject may have been previously administered an immunogenic composition. The subject may not have been previously administered an immunogenic composition. Alternatively, the subject will be administered an immunogenic composition.

Circulating tumor DNA can be analyzed at various time points. For example, prior to, immediately prior to, or after cancer treatment (e.g., administration with an immunogenic composition, such as a tumor-specific neoantigen composition). Circulating tumor DNA can be analyzed at more than one time point, for example at the end, right before the end of one or more additional cycles of treatment (e.g., administration with an immunogenic composition, such as a tumor-specific neoantigen composition). Circulating tumor DNA can analyzed about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days or longer after administration of an immunogenic composition. Circulating tumor DNA can analyzed about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks or longer after administration of an immunogenic composition. Circulating tumor DNA can analyzed about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months or longer after administration of an immunogenic composition. Circulating DNA can be analyzed about 1 year, about 1.5 years, about 2 years, about 2.5 years, about 3 years, about 3.5 years, about 4 years, about 4.5 years, about 5 years or longer after administration of an immunogenic composition.

One or more immunogenic compositions (e.g., about 1, about 2, about 3, about 4, about 5, about 6, about 7, about 8, about 9, about 10 or more) can be generated based on the information derived from the circulating tumor DNA. The immunogenic compositions can be modulated over time based on the information derived from the circulating tumor DNA. For example, a personalized immunogenic composition can be generated based on monitoring a patient during treatment and adjusting the immunogenic composition accordingly.

The cancer can be any solid tumor or any hematological tumor. The methods disclosed herein are preferably suited for solid tumors. The tumor can be a primary tumor (e.g., a tumor that is at the original site where the tumor first arose). Solid tumors can include, but are not limited to, breast cancer tumors, ovarian cancer tumors, prostate cancer tumors, lung cancer tumors, kidney cancer tumors, gastric cancer tumors, testicular cancer tumors, head and neck cancer tumors, pancreatic cancer tumors, brain cancer tumors, and melanoma tumors. Hematological tumors can include, but are not limited to, tumors from lymphomas (e.g., B cell lymphomas) and leukemias (e.g., acute myelogenous leukemia, chronic myelogenous leukemia, chronic lymphocytic leukemia, and T cell lymphocytic leukemia).

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In some embodiments, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

Immunogenic compositions stimulate a subject's immune system, especially the response of specific CD8+ T cells or CD4+ T cells. Interferon gamma produced by CD8+ and T helper CD4+ cells regulate the expression of PD-L1. PD-L1 expression in tumor cells is upregulated when attacked by T cells. Thus, tumor vaccines may induce the production of specific T cells and simultaneously upregulate the expression of PD-L1, which may limit the efficacy of the immunogenic composition. In addition, while the immune system is activated, the expression of T cell surface reporter CTLA-4 is correspondingly increased, which binds with the ligand B7-1/B7-2 on antigen-presenting cells and plays an immunosuppressant effect. Thus, in some instances, the subject may also be administered an anti-immunosuppressive or immunostimulatory agent, such as a checkpoint inhibitor. Checkpoint inhibitors can include, but are not limited to, anti-CTL4-A antibodies, anti-PD-1 antibodies and anti-PD-L1 antibodies. These checkpoint inhibitors bind to the immune checkpoint proteins of T cells to remove the inhibition of T cell function by tumor cells. Blockade of CTLA-4 or PD-L1 by antibodies can enhance the immune response to cancerous cells in the patient. CTLA-4 has been shown to be effective when following a vaccination protocol.

An immunogenic composition comprising one or more tumor-specific neoantigens can be administered to a subject that has been diagnosed with cancer, is already suffering from cancer, has recurrent cancer (i.e., relapse), or is at risk of developing cancer. An immunogenic composition comprising one or more tumor-specific neoantigens can be administered to a subject that is resistant to other forms of cancer treatment (e.g., chemotherapy, immunotherapy, or radiation). An immunogenic composition comprising one or more tumor-specific neoantigens can be administered to the subject prior to other standard of care cancer therapies (e.g., chemotherapy, immunotherapy, or radiation). An immunogenic composition comprising one or more tumor-specific neoantigens can be administered to the subject concurrently, after, or in combination with other standard of care cancer therapies (e.g., chemotherapy, immunotherapy, or radiation).

The subject can be a human, dog, cat, horse, or any animal for which a tumor specific response is desired.

The immunogenic composition is administered to the subject in an amount sufficient to elicit an immune response to the tumor-specific neoantigen and to destroy, or at least partially arrest, symptoms and/or complications. In embodiments, the immunogenic composition can provide a long-lasting immune response. A long-lasting immune response can be established by administering a boosting dose of the immunogenic composition to the subject. The immune response to the immunogenic composition can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000%. A second boosting dose may increase the immune response by at least 50%, at least 100%, at least 200%, at least 300%, at least 400%, at least 500%, or at least 1000%. A third boosting dose may increase the immune response by at least 50%, at least 100%, at least 200%, at least 300%, at least 400%, at least 500%, or at least 1000%.

An amount adequate to elicit an immune response is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that immunogenic compositions can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of these immunogenic compositions.

The immunogenic composition comprising one or more tumor-specific neoantigens can be administered to the subject alone or in combination with other therapeutic agents. The therapeutic agent can be, for example, a chemotherapeutic agent, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. The subject may be administered a small molecule, or targeted therapy (e.g. kinase inhibitor). The subject may be further administered an anti-CTLA-4 antibody or anti-PD-1 antibody or anti-PD-L1 antibody. Blockade of CTLA-4 or PD-L1 by antibodies can enhance the immune response to cancerous cells in the patient.

### C. Immunogenic Composition

The invention further relates to immunogenic compositions (e.g. an antigenic composition or a subject specific composition). The invention particularly relates to personalized (i.e., subject-specific) immunogenic compositions (e.g., a cancer vaccine) comprising one or more tumor-specific antigens selected using the methods described herein. Such immunogenic compositions can be formulated according to standard procedures in the art. The immunogenic composition is capable of raising a specific immune response.

The immunogenic composition can be formulated so that the selection and number of tumor-specific neoantigens is tailored to the subject's particular cancer. For example, the selection of the tumor-specific neoantigens can be dependent on the specific type of cancer, the status of the cancer, the immune status of the subject, and the MHC-type of the subject.

The immunogenic composition can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 37, 37, 38, 39, 40, 41, 42, 43, 44, 45, 46, 47, 48, 49, 50 or more tumor-specific neoantigens. The immunogenic composition can contain about 10-20 tumor-specific neoantigens, about 10-30 tumor-specific neoantigens, about 10-40 tumor-specific neoantigens, about 10-50 tumor-specific neoantigens, about 10-60 tumor-specific neoantigens, about 10-70 tumor-specific neoantigens, about 10-80 tumor-specific neoantigens, about 10-90 tumor-specific neoantigens, or about 10-100 tumor-specific neoantigens. In one aspect, the immunogenic composition comprises at least about 10 tumor-specific neoantigens. In another aspect, the immunogenic composition comprises at least about 20 tumor-specific neoantigens.

The immunogenic composition can further comprise natural or synthetic antigens. The natural or synthetic antigens can increase the immune response. Exemplary natural or synthetic antigens include, but are not limited to, pan-DR epitope (PADRE) and tetanus toxin antigen.

The immunogenic composition can be in any form, for example a synthetic long peptide, RNA, DNA, a cell, a dendritic cell, a nucleotide sequence, a polypeptide sequence, a plasmid, or a vector.

Tumor-specific neoantigens can also be included in viral vector-based vaccine platforms, such as vaccinia, fowlpox, self-replicating alphavims, marabavirus, adenovirus (See, e.g., Tatsis et al., Molecular Therapy, 10:616-629 (2004)), or lentivirus, including but not limited to second, third or hybrid second/third generation lentivirus and recombinant lentivirus of any generation designed to target specific cell types or receptors (See, e.g., Hu et al., Immunol Rev., 239(1): 45-61 (2011), Sakma et al, Biochem J., 443(3):603-18 (2012)). Dependent on the packaging capacity of the above-mentioned viral vector-based vaccine platforms, this approach can deliver one or more nucleotide sequences that encode one or more tumor-specific neoantigen peptides. The sequences may be flanked by non-mutated sequences, may be separated by linkers or may be preceded with one or more sequences targeting a subcellular compartment (See, e.g., Gros et al., Nat Med., 22 (4):433-8 (2016), Stronen et al., Science., 352(6291): 1337-1341 (2016), Lu et al., Clin Cancer Res., 20(13):3401-3410 (2014)). Upon introduction into a host, infected cells express the one or more tumor-specific neoantigens, and thereby elicit a host immune (e.g., CD8+ or CD4+) response against the one or more tumor-specific neoantigens. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens that will be apparent to those skilled in the art from the description herein may also be used.

The immunogenic composition can contain individualized components, according to the personal needs of the particular subject.

The immunogenic composition described herein can further comprise an adjuvant. Adjuvants are any substance whose admixture into an immunogenic composition increases, or otherwise enhances and/or boosts, the immune response to a tumor-specific neoantigen, but when the substance is administered alone does not generate an immune response to a tumor-specific neoantigen. The adjuvant preferably generates an immune response to the neoantigen and does not produce an allergy or other adverse reaction. It is contemplated herein that the immunogenic composition can be administered before, together, concomitantly with, or after administration of the immunogenic composition.

Adjuvants can enhance an immune response by several mechanisms including, e.g., lymphocyte recruitment, stimulation of B and/or T cells, and stimulation of macrophages. When an immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), AS03 (Glaxo SmithKline), AS04 (Glaxo SmithKline), polysorbate 80 (Tween 80; ICL Americas, Inc.), imidazopyridine compounds (see, International Application No. PCT/US2007/064857, published as International Publication No. WO2007/109812), imidazoquinoxaline compounds (see, International Application No. PCT/US2007/064858, published as International Publication No. WO2007/109813) and saponins, such as QS21 (see, Kensil et al, in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)).

CpG immunostimulatory oligonucleotides have also been reported to enhance the

effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I.C)(e.g. polyi:CI2U), poly ICLC, non-CpG bacterial DNA or RNA as well as immunoactive small molecules and antibodies such as cyclophosphamide, sunitmib, bevacizumab, Celebrex (celecoxib), NCX-4016, sildenafil, tadalafil, vardenafil, sorafinib, XL-999, CP-547632, pazopamb, ZD2171, AZD2171, ipilimumab, tremelimumab, and SC58175, which may act therapeutically and/or as an adjuvant. In embodiments, Poly ICLC is an adjuvant.

The immunogenic compositions can comprise one or more tumor-specific neoantigens described herein alone or together with a pharmaceutically acceptable carrier. Suspensions or dispersions of one or more tumor-specific neoantigens, especially isotonic aqueous suspensions, dispersions, or amphiphilic solvents can be used. The immunogenic compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

An excipient can be present independently of an adjuvant. The function of an excipient can be, for example, to increase the molecular weight of the immunogenic composition, to increase activity or immunogenicity, to confer stability, to increase the biological activity, or to increase serum-half life. An excipient can also be used to aid presentation of the one or more tumor-specific neoantigens to T-cells (e.g., CD4+ or CD8+ T-cells). The excipient can be a carrier protein such as, but not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. Alternatively, the carrier can be dextran, for example Sepharose.

Cytotoxic T-cells recognize an antigen in the form of a peptide bound to an MHC molecule, rather than the intact foreign antigen itself. The MHC molecule itself is located at the cell surface of an antigen presenting cell. Thus, an activation of cytotoxic T-cells is possible if a trimeric complex of peptide antigen, MHC molecule, and antigen-presenting cell (APC) is present. It may enhance the immune response if not only the one or more tumor-specific antigens are used for activation of cytotoxic T-cells, but if additional APCs with the respective MHC molecule are added. Thus, in some embodiments an immunogenic composition additionally contains at least one APC.

The immunogenic composition can comprise an acceptable carrier (e.g., an aqueous carrier). A variety of aqueous carriers can be used, e.g., water, buffered water, 0.9% saline, 0.3% glycine, hyaluronic acid and the like. These compositions can be sterilized by conventional, well-known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Neoantigens can also be administered via liposomes, which target them to a particular cell tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., An. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

As an alternative method for targeting immune cells, components of the immunogenic composition, such as an antigen (i.e., tumor-specific neoantigen), ligand, or adjuvant (e.g., TLR) can be incorporated into an poly(lactic-co-glycolic) microspheres. The poly(lactic-co-glycolic) microspheres can entrap components of the immunogenic composition as an endosomal delivery device.

For therapeutic or immunization purposes, nucleic acids encoding a tumor-specific neoantigen described herein can also be administered to the patient. A number of methods are conveniently used to deliver the nucleic acids to the patient. For example, the nucleic acid can be delivered directly, as “naked DNA.” This approach is described, for example, in Wolff et al., Science 247: 1465-1468 (1990), as well as U.S. Pat. Nos. 5,580,859 and 5,589,466. The nucleic acids can also be administered using ballistic delivery as described, for example, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation. The nucleic acids can also be delivered complexed to cationic compounds, such as cationic lipids.

The immunogenic compositions provided herein can be administered to the subject by various routes including, but not limited to, oral, intradermal, intratumoral, intramuscular, intraperitoneal, intravenous, topical, subcutaneous, percutaneous, intranasal and inhalation routes, and via scarification (scratching through the top layers of skin, e.g., using a bifurcated needle). The immunogenic composition can be administered at the tumor site to induce a local immune response to the tumor.

The dosage of the one or more tumor-specific neoantigens may depend upon the type of composition and upon the subject's age, weight, body surface area, individual condition, the individual pharmacokinetic data, and the mode of administration.

Also disclosed herein is a method of manufacturing an immunogenic composition comprising one or more tumor-specific neoantigens selected by performing the steps of the methods disclosed herein. An immunogenic composition as described herein can be manufactured using methods known in the art. For example, a method of producing a tumor-specific neoantigen or a vector (e.g., a vector including at least one sequence encoding one or more tumor-specific neoantigens) disclosed herein can include culturing a host cell under conditions suitable for expressing the neoantigen or vector, wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NSO cell, yeast, or a HEK293 cell. Host cells can be transformed with one or more polynucleotides comprising at least one nucleic acid sequence that encodes one or more tumor-specific neoantigens or vector disclosed herein. In certain embodiments the isolated polynucleotide can be cDNA.

### D. Samples

The methods disclosed herein comprise obtaining circulating tumor DNA from a biological sample from a subject. The subject has been previously administered or will be prospectively administered with an immunogenic composition (i.e., a tumor-specific neoantigen or an antigen-based vaccine). The biological sample can be obtained from human or non-human subjects. Preferentially, the biological sample is obtained from a human. The biological sample can be obtained from a variety of biological sources.

Various assays (e.g., sequencing assays) can be used to detect circulating tumor DNA. The methods provided herein can comprise isolation and analysis of circulating tumor DNA from the blood (e.g., plasma or serum) of a subject of interest (i.e., a subject having cancer, a subject in remission of cancer, or a subject suspected of having cancer). The method can comprise isolating plasma and circulating tumor DNA from intact cell-depleted blood. The method can comprise centrifugation to generate plasma and extraction of nucleic acids from plasma.

The circulating tumor DNA can be from a bodily fluid (e.g., a blood sample) of a subject of interest. The circulating tumor DNA can be obtained from plasma fraction, serum fraction, or both, of the blood sample. In some embodiments, the bodily sample is whole blood, serum, plasma, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine, or any combination thereof. In some embodiment, the circulating DNA is obtained from blood and fractions thereof. A sample can be in the form originally isolated from a subject or can be subjected to further processing to remove or add components, such as cells, or enrich for one component relative to another. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may not have cancer or a detectable cancer symptom. The subject may have been treated with one or more cancer therapy, e.g., any one or more of chemotherapies, antibodies, vaccines or biologics. The subject may be in remission. The subject may be suspected to have cancer or any cancer-associated genetic mutations.

The biological sample can be obtained from a subject by any means including, but not limited to, tumor biopsy, needle aspirate, scraping, surgical excision, surgical incision, venipuncture, or other means known in the art. Those skilled in the art will recognize other suitable techniques for obtaining biological samples.

The biological sample can be obtained from the subject in a single procedure. The biological sample can be obtained from the subject repeatedly over a period of time. For example, the biological sample may be obtained once a day, once a week, monthly, biannually, or annually. Obtaining numerous samples over a period of time can be useful to identify and select new tumor-specific neoantigens.

The biological sample can be obtained from tumor draining veins (e.g., immediately downstream or locoregional). The biological sample can be obtained from veins that drain in close proximity to the tumor of interest, thus allowing for monitoring of the circulating tumor DNA that has not been subject to filtering through the liver and/or other organs that may alter the circulating tumor DNA.

The biological sample can be obtained from lymph downstream (e.g., immediately downstream) from a tumor. The biological sample can be obtained from lymphatic vessels, for example by cannulation, that drain in close proximity to the tumor of interest, thus allowing for monitoring of the circulating tumor DNA that has not been subject to dilution in venous blood, e.g. through the thoracic duct; through the thoracic and right lymphatic ducts.

The circulating tumor DNA can be isolated from bodily fluids (e.g., plasma) through a fractionation or partitioning step in which circulating tumor DNA, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. After such processing, samples can include various forms of nucleic acid including double stranded DNA and single stranded DNA. In some embodiments, single stranded DNA can be converted to double stranded forms so they are included in subsequent processing and analysis steps.

### E. Implementation on a Computer Environment

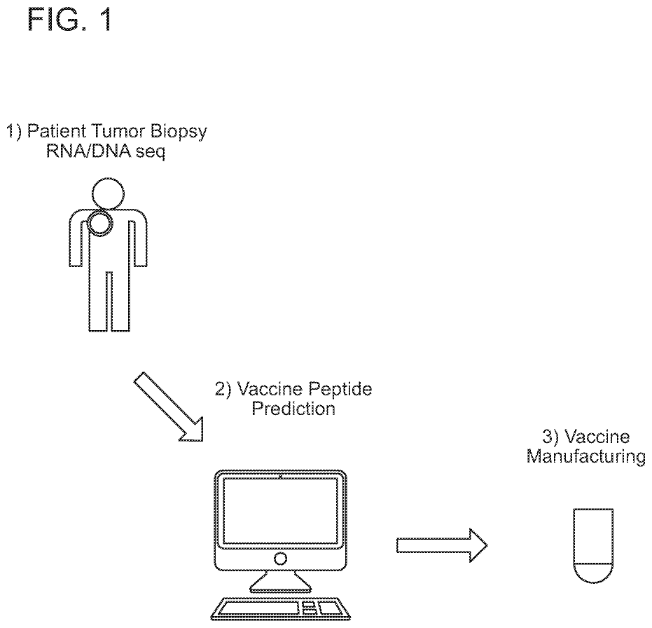

All or any portion of the above described can be implemented on a computing environment such as those illustrated in FIGS. 1-3. FIG. 1 illustrates an exemplary provider network (or “service provider system”) environment according to some embodiments. A provider network 900 may provide resource virtualization to customers via one or more virtualization services 910 that allow customers to purchase, rent, or otherwise obtain instances 912 of virtualized resources, including but not limited to computation and storage resources, implemented on devices within the provider network or networks in one or more data centers. Local Internet Protocol (IP) addresses 916 may be associated with the resource instances 912, the local IP addresses are the internal network addresses of the resource instances 912 on the provider network 900. In some embodiments, the provider network 900 may also provide public IP addresses 914 and/or public IP address ranges (e.g., Internet Protocol version 4 (IPv4) or Internet Protocol version 6 (IPv6) addresses) that customers may obtain from the provider 900.

Conventionally, the provider network 900, via the virtualization services 910, may allow a customer of the service provider (e.g., a customer that operates one or more client networks 950A-950C including one or more customer device(s) 952) to dynamically associate at least some public IP addresses 914 assigned or allocated to the customer with particular resource instances 912 assigned to the customer. The provider network 900 may also allow the customer to remap a public IP address 914, previously mapped to one virtualized computing resource instance 912 allocated to the customer, to another virtualized computing resource instance 912 that is also allocated to the customer. Using the virtualized computing resource instances 912 and public IP addresses 914 provided by the service provider, a customer of the service provider such as the operator of customer network(s) 950A-950C may, for example, implement customer-specific applications and present the customer's applications on an intermediate network 940, such as the Internet. Other network entities 920 on the intermediate network 940 may then generate traffic to a destination public IP address 914 published by the customer network(s) 950A-950C; the traffic is routed to the service provider data center, and at the data center is routed, via a network substrate, to the local IP address 916 of the virtualized computing resource instance 912 currently mapped to the destination public IP address 914. Similarly, response traffic from the virtualized computing resource instance 912 may be routed via the network substrate back onto the intermediate network 940 to the source entity 920.

Local IP addresses, as used herein, refer to the internal or “private” network addresses, for example, of resource instances in a provider network. Local IP addresses can be within address blocks reserved by Internet Engineering Task Force (IETF) Request for Comments (RFC) 1918 and/or of an address format specified by IETF RFC 4193 and may be mutable within the provider network. Network traffic originating outside the provider network is not directly routed to local IP addresses; instead, the traffic uses public IP addresses that are mapped to the local IP addresses of the resource instances. The provider network may include networking devices or appliances that provide network address translation (NAT) or similar functionality to perform the mapping from public IP addresses to local IP addresses and vice versa.

Public IP addresses are Internet mutable network addresses that are assigned to resource instances, either by the service provider or by the customer. Traffic routed to a public IP address is translated, for example via 1:1 NAT, and forwarded to the respective local IP address of a resource instance.

Some public IP addresses may be assigned by the provider network infrastructure to particular resource instances; these public IP addresses may be referred to as standard public IP addresses, or simply standard IP addresses. In some embodiments, the mapping of a standard IP address to a local IP address of a resource instance is the default launch configuration for all resource instance types.

At least some public IP addresses may be allocated to or obtained by customers of the provider network 900; a customer may then assign their allocated public IP addresses to particular resource instances allocated to the customer. These public IP addresses may be referred to as customer public IP addresses, or simply customer IP addresses. Instead of being assigned by the provider network 900 to resource instances as in the case of standard IP addresses, customer IP addresses may be assigned to resource instances by the customers, for example via an API provided by the service provider. Unlike standard IP addresses, customer IP addresses are allocated to customer accounts and can be remapped to other resource instances by the respective customers as necessary or desired. A customer IP address is associated with a customer's account, not a particular resource instance, and the customer controls that IP address until the customer chooses to release it. Unlike conventional static IP addresses, customer IP addresses allow the customer to mask resource instance or availability zone failures by remapping the customer's public IP addresses to any resource instance associated with the customer's account. The customer IP addresses, for example, enable a customer to engineer around problems with the customer's resource instances or software by remapping customer IP addresses to replacement resource instances.

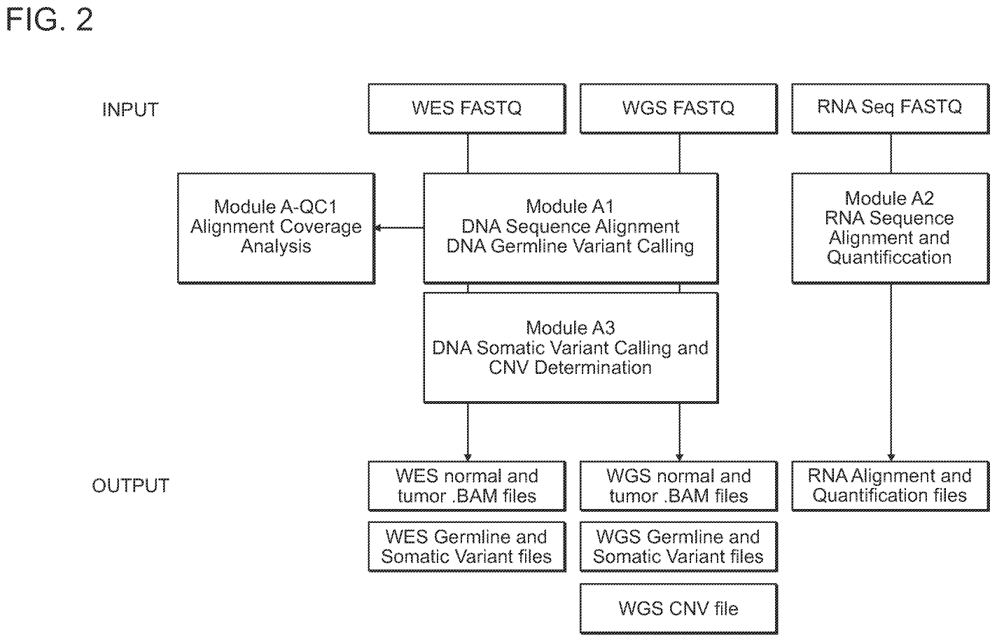

FIG. 2 is a block diagram of an example provider network that provides a storage service and a hardware virtualization service to customers, according to some embodiments. Hardware virtualization service 1020 provides multiple computation resources 1024 (e.g., VMs) to customers. The computation resources 1024 may, for example, be rented or leased to customers of the provider network 1000 (e.g., to a customer that implements customer network 1050). Each computation resource 1024 may be provided with one or more local IP addresses. Provider network 1000 may be configured to route packets from the local IP addresses of the computation resources 1024 to public Internet destinations, and from public Internet sources to the local IP addresses of computation resources 1024.

Provider network 1000 may provide a customer network 1050, for example coupled to intermediate network 1040 via local network 1056, the ability to implement virtual computing systems 1092 via hardware virtualization service 1020 coupled to intermediate network 1040 and to provider network 1000. In some embodiments, hardware virtualization service 1020 may provide one or more APIs 1002, for example a web services interface, via which a customer network 1050 may access functionality provided by the hardware virtualization service 1020, for example via a console 1094 (e.g., a web-based application, standalone application, mobile application, etc.). In some embodiments, at the provider network 1000, each virtual computing system 1092 at customer network 1050 may correspond to a computation resource 1024 that is leased, rented, or otherwise provided to customer network 1050.

From an instance of a virtual computing system 1092 and/or another customer device 1090 (e.g., via console 1094), the customer may access the functionality of storage service 1010, for example via one or more APIs 1002, to access data from and store data to storage resources 1018A-1018N of a virtual data store 1016 (e.g., a folder or “bucket”, a virtualized volume, a database, etc.) provided by the provider network 1000. In some embodiments, a virtualized data store gateway (not shown) may be provided at the customer network 1050 that may locally cache at least some data, for example frequently-accessed or critical data, and that may communicate with storage service 1010 via one or more communications channels to upload new or modified data from a local cache so that the primary store of data (virtualized data store 1016) is maintained. In some embodiments, a user, via a virtual computing system 1092 and/or on another customer device 1090, may mount and access virtual data store 1016 volumes via storage service 1010 acting as a storage virtualization service, and these volumes may appear to the user as local (virtualized) storage 1098.

While not shown in FIG. 2, the virtualization service(s) may also be accessed from resource instances within the provider network 1000 via API(s) 1002. For example, a customer, appliance service provider, or other entity may access a virtualization service from within a respective virtual network on the provider network 1000 via an API 1002 to request allocation of one or more resource instances within the virtual network or within another virtual network.

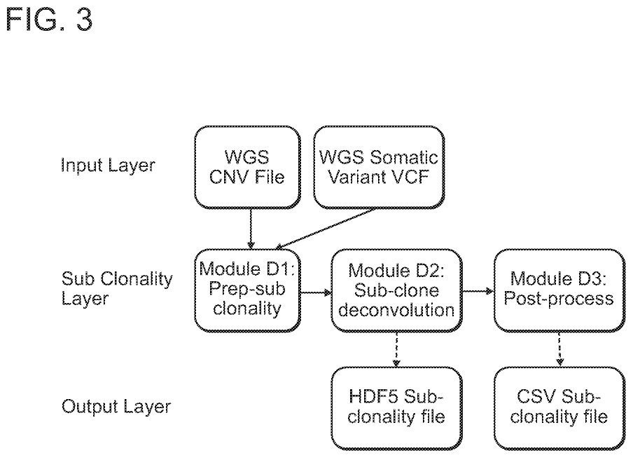

In some embodiments, a system that implements a portion or all of the techniques described herein may include a general-purpose computer system that includes or is configured to access one or more computer-accessible media, such as the computer system 1100 illustrated in FIG. 3. In the illustrated embodiment, a computer system 1100 includes one or more processors 1110 coupled to a system memory 1120 via an input/output (I/O) interface 1130. The computer system 1100 further includes a network interface 1140 coupled to I/O interface 1130.

While FIG. 3 shows a computer system 1100 as a single computing device, in various embodiments a computer system 1100 may include one computing device or any number of computing devices configured to work together as a single computer system 1100.

In various embodiments, computer system 1100 may be a uniprocessor system including one processor 1110, or a multiprocessor system including several processors 1110 (e.g., two, four, eight, or another suitable number). Processors 1110 may be any suitable processors capable of executing instructions. For example, in various embodiments, processors 1110 may be general-purpose or embedded processors implementing any of a variety of instruction set architectures (ISAs), such as the x86, ARM, PowerPC, SPARC, or MIPS ISAs, or any other suitable ISA. In multiprocessor systems, each of processors 1110 may commonly, but not necessarily, implement the same ISA.

System memory 1120 may store instructions and data accessible by processor(s) 1110. In various embodiments, system memory 1120 may be implemented using any suitable memory technology, such as random-access memory (RAM), static RAM (SRAM), synchronous dynamic RAM (SDRAM), nonvolatile/Flash-type memory, or any other type of memory. In the illustrated embodiment, program instructions and data implementing one or more desired functions, such as those methods, techniques, and data described above are shown stored within system memory 1120 as enzyme-substrate predictor service code 1125 and data 1126.

In one embodiment, the I/O interface 1130 may be configured to coordinate I/O traffic between processor 1110, system memory 1120, and any peripheral devices in the device, including network interface 1140 or other peripheral interfaces. In some embodiments, the I/O interface 1130 may perform any necessary protocol, timing or other data transformations to convert data signals from one component (e.g., system memory 1120) into a format suitable for use by another component (e.g., processor 1110). In some embodiments, the I/O interface 1130 may include support for devices attached through various types of peripheral buses, such as a variant of the Peripheral Component Interconnect (PCI) bus standard or the Universal Serial Bus (USB) standard, for example. In some embodiments, the function of the I/O interface 1130 may be split into two or more separate components, such as a north bridge and a south bridge, for example. Also, in some embodiments some or all of the functionality of the I/O interface 1130, such as an interface to system memory 1120, may be incorporated directly into the processor 1110.

The network interface 1140 may be configured to allow data to be exchanged between a computer system 1100 and other devices 1160 attached to a network or networks 1150. In various embodiments, the network interface 1140 may support communication via any suitable wired or wireless general data networks, such as types of Ethernet network, for example. Additionally, the network interface 1140 may support communication via telecommunications/telephony networks such as analog voice networks or digital fiber communications networks, via storage area networks (SANs) such as Fibre Channel SANs, or via I/O any other suitable type of network and/or protocol.

In some embodiments, a computer system 1100 includes one or more offload cards 1170 (including one or more processors 1175, and possibly including the one or more network interfaces 1140) that are connected using an I/O interface 1130 (e.g., a bus implementing a version of the Peripheral Component Interconnect Express (PCI-E) standard, or another interconnect such as a QuickPath interconnect (QPI) or UltraPath interconnect (UPI)). For example, in some embodiments the computer system 1100 may act as a host electronic device (e.g., operating as part of a hardware virtualization service) that hosts compute instances, and the one or more offload cards 1170 execute a virtualization manager that can manage compute instances that execute on the host electronic device. As an example, in some embodiments the offload card(s) 1170 can perform compute instance management operations such as pausing and/or un-pausing compute instances, launching and/or terminating compute instances, performing memory transfer/copying operations, etc. These management operations may, in some embodiments, be performed by the offload card(s) 1170 in coordination with a hypervisor (e.g., upon a request from a hypervisor) that is executed by the other processors 1110A-1110N of the computer system 1100. However, in some embodiments the virtualization manager implemented by the offload card(s) 1170 can accommodate requests from other entities (e.g., from compute instances themselves), and may not coordinate with (or service) any separate hypervisor.

In some embodiments, system memory 1120 may be one embodiment of a computer-accessible medium configured to store program instructions and data as described above. However, in other embodiments, program instructions and/or data may be received, sent, or stored upon different types of computer-accessible media. Generally speaking, a computer-accessible medium may include non-transitory storage media or memory media such as magnetic or optical media, e.g., disk or DVD/CD coupled to a computer system 1100 via I/O interface 1130. A non-transitory computer-accessible storage medium may also include any volatile or non-volatile media such as RAM (e.g., SDRAM, double data rate (DDR) SDRAM, SRAM, etc.), read only memory (ROM), etc., that may be included in some embodiments of a computer system 1100 as system memory 1120 or another type of memory. Further, a computer-accessible medium may include transmission media or signals such as electrical, electromagnetic, or digital signals, conveyed via a communication medium such as a network and/or a wireless link, such as may be implemented via a network interface 1140.

Various embodiments discussed or suggested herein can be implemented in a wide variety of operating environments, which in some cases can include one or more user computers, computing devices, or processing devices which can be used to operate any of a number of applications. User or client devices can include any of a number of general-purpose personal computers, such as desktop or laptop computers running a standard operating system, as well as cellular, wireless, and handheld devices running mobile software and capable of supporting a number of networking and messaging protocols. Such a system also can include a number of workstations running any of a variety of commercially available operating systems and other known applications for purposes such as development and database management. These devices also can include other electronic devices, such as dummy terminals, thin-clients, gaming systems, and/or other devices capable of communicating via a network.

Most embodiments utilize at least one network that would be familiar to those skilled in the art for supporting communications using any of a variety of widely-available protocols, such as Transmission Control Protocol/Internet Protocol (TCP/IP), File Transfer Protocol (FTP), Universal Plug and Play (UPnP), Network File System (NFS), Common Internet File System (CIFS), Extensible Messaging and Presence Protocol (XMPP), AppleTalk, etc. The network(s) can include, for example, a local area network (LAN), a wide-area network (WAN), a virtual private network (VPN), the Internet, an intranet, an extranet, a public switched telephone network (PSTN), an infrared network, a wireless network, and any combination thereof.

In embodiments utilizing a web server, the web server can run any of a variety of server or mid-tier applications, including HTTP servers, File Transfer Protocol (FTP) servers, Common Gateway Interface (CGI) servers, data servers, Java servers, business application servers, etc. The server(s) also may be capable of executing programs or scripts in response requests from user devices, such as by executing one or more Web applications that may be implemented as one or more scripts or programs written in any programming language, such as Java®, C, C#or C++, or any scripting language, such as Perl, Python, PHP, or TCL, as well as combinations thereof. The server(s) may also include database servers, including without limitation those commercially available from Oracle®, Microsoft®, Sybase®, IBM®, etc. The database servers may be relational or non-relational (e.g., “NoSQL”), distributed or non-distributed, etc.

Environments disclosed herein can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and/or at least one output device (e.g., a display device, printer, or speaker). Such a system may also include one or more storage devices, such as disk drives, optical storage devices, and solid-state storage devices such as random-access memory (RAM) or read-only memory (ROM), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, Electrically Erasable Programmable Read-Only Memory (EEPROM), flash memory or other memory technology, Compact Disc-Read Only Memory (CD-ROM), Digital Versatile Disk (DVD) or other optical storage, magnetic cassettes, magnetic tape, magnetic disk storage or other magnetic storage devices, or any other medium which can be used to store the desired information and which can be accessed by a system device. Based on the disclosure and teachings provided herein, a person of ordinary skill in the art will appreciate other ways and/or methods to implement the various embodiments.

In the preceding description, various embodiments are described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Bracketed text and blocks with dashed borders (e.g., large dashes, small dashes, dot-dash, and dots) are used herein to illustrate optional operations that add additional features to some embodiments. However, such notation should not be taken to mean that these are the only options or optional operations, and/or that blocks with solid borders are not optional in certain embodiments.

Reference numerals with suffix letters may be used to indicate that there can be one or multiple instances of the referenced entity in various embodiments, and when there are multiple instances, each does not need to be identical but may instead share some general traits or act in common ways. Further, the particular suffixes used are not meant to imply that a particular amount of the entity exists unless specifically indicated to the contrary. Thus, two entities using the same or different suffix letters may or may not have the same number of instances in various embodiments.

References to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Moreover, in the various embodiments described above, unless specifically noted otherwise, disjunctive language such as the phrase “at least one of A, B, or C” is intended to be understood to mean either A, B, or C, or any combination thereof (e.g., A, B, and/or C). As such, disjunctive language is not intended to, nor should it be understood to, imply that a given embodiment requires at least one of A, at least one of B, or at least one of C to each be present

### F. Definitions

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in subjects in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, Hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu. Hormone receptor positive breast cancer refers to breast cancer that is positive for at least one of the following: ER or PR, and negative for Her2/neu (HER2).

The term “neoantigen” as used herein refers to an antigen that has at least one alteration that makes it distinct from the corresponding parent antigen, e.g., via mutation in a tumor cell or post-translational modification specific to a tumor cell. A mutation can include a frameshift, indel, missense or nonsense substitution, splice site alteration, genomic rearrangement or gene fusion, or any genomic expression alteration giving rise to a neoantigen. A mutation can include a splice mutation. Post-translational modifications specific to a tumor cell can include aberrant phosphorylation. Post-translational modifications specific to a tumor cell can also include a proteasome-generated spliced antigen. See, Lipe et al., Science, 354(6310):354:358 (2016). In general, point mutations account for about 95% mutations in tumors, and indels and frame-shift mutations account for the rest. See, Snyder et al., N Engl J Med., 371:2189-2199 (2014).

As used herein the term “tumor-specific neoantigen” is a neoantigen present in a subject's tumor cell or tissue, but not in the subject's normal cell or tissue.

The term “germline sibling” as used herein refers to germline antigens that represent the un-mutated peptide equivalent of a corresponding neoantigen.

The term “neural network” as used herein refers to a machine-learning model for classification or regression consisting of multiple layers of linear transformations followed by element-wise nonlinearities typically trained via stochastic gradient descent and back-propagation.

The term “propagating the labelled somatic variants” refers to training one of the machine learning ensemble models on the coding region for deeply sampled whole genome sequence data and then using the predictions from the machine learning model to label the non-coding regions. Later, the deep whole genome sequence data (˜400x) is sub-sampled into lower depth whole genome sequence data (˜200x), a new machine learning model is trained on the sub-sampled data, and the performance of this model is evaluated on the labels derived from the first step.

The term “subject” as used herein refers to any animal, such as any mammal, including but not limited to, humans, non-human primates, rodents, and the like. In some embodiments, the mammal is a mouse. In some embodiments, the mammal is a human.

The term “tumor cell” as used herein refers to any cell that is a cancer cell or is derived from a cancer cell. The term “tumor cell” can also refer to a cell that exhibits cancer-like properties, e.g., uncontrollable reproduction, resistance to anti-growth signals, ability to metastasize, and loss of ability to undergo programed cell death.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### 7. EXAMPLES

### Example 1. Blood Sample Collection

Blood samples will be collected at minimum of once pre-treatment, and followed by serial collections guided by the monitoring-frequency of the vaccine efficacy and disease progression.

Pre-treatment specimen collection: 10 ml volume of blood will be collected from the patient within a 5-day interval of the biopsy collected for paired genomic (including but not limited to WGS/WES) tumor-normal sequencing, and within 2-4 weeks before the treatment (vaccine) is administered.

Post-treatment specimen collection: Serial collection of blood specimens will be performed based on medically informed indications of disease progression, clinically observed response to the vaccine, or at regularly scheduled intervals when patients are scheduled to get follow-up vaccines of the original regimen and at minimum once at the time of vaccine administration and once within 10 weeks of vaccine administration.

### Example 2. Sample Processing, cfDNA Extraction and ctDNA Sequencing

Blood samples will be processed within 4 hours and cell-free DNA (cfDNA) isolated from plasma using standard methods (e.g., Qiagen DNeasy kit, QIAmp kit, or Quick-cfDNA kit). In the pre-treatment specimen processing, separate to the cfDNA isolation from plasma, peripheral blood lymphocytes from the first centrifugation step in cfDNA isolation are also used to extract germline genomic DNA, which is used as the matched normal for the genomic analysis.

Before library construction, a minimum yield of 20 ng of DNA per blood aliquot is ensured.

ctDNA sequencing will be performed using Illumina NexSeq or NovaSeq600 to obtain paired-end DNA reads from the DNA library. The sequence reads will be delivered to the bioinformatics pipelines in a pair of FASTQ files or in an interleaved FASTQ file. Targeted (eg PARE) will be performed to track specific mutations/genomic events. A pre-determined panel can be used, such as Guardant (N=73 genes) or Circulogene (N=50 genes). Alternatively we may ‘pre-empt’ expected evasion routes and cast a wider net for shotgun sequencing instead of targeted sequencing.

ctDNA bioinformatics processing. After removing terminal adaptor sequences and filtering out poor quality data, reads will be mapped to the reference human genome using BWA-mem or an equivalent method. Variants of interest will be compiled with their allelic fractions recorded. We will either map the ctDNA reads to reference and get ‘pileups’ at each ‘different from reference’ site, or follow the paired tumor-normal approach for variant calling where the tumor files are from ctDNA sequencing and the normal is the default WGS normal sequencing.

Aligning ctDNA reads to a reference. The FASTQ files will contain nucleotide sequences in the individual reads. Some of these reads will be ctDNA and some will be germline tissue, with the ratio depending on the quality of the ctDNA isolation step. A genome reference, such as GRCh38 will be aligned to the reads. Bioinformatic software, such as DRAGEN, will be used to look for exact matches between the short reads and the reference genome, using Smith-Waterman Alignment. A bioinformatics software, such as DRAGEN, will be used to perform local optimization and variant calling for reads that do not have exact matches from SWA.

ctDNA data analysis. Identified variants in the ctDNA will be cross-referenced to the variants identified from WGS and/or WES. Variants identified from WGS/WES sequencing will be assigned to tumor sub-clones, either via bulk deconvolution methods (such as PyClone in ref) or single-cell sequencing. Variants from the ctDNA will be assigned the same clonal distribution as the same variant identified from WGS/WES.

Peptides associated to transcribed RNA that contain the ctDNA mutations will be scored by a function that considers the cellular prevalence of both the variants present in both the ctDNA and WES/WGS, as well as variants that appear only in the WES/WGS.

1. A method for evaluating the efficacy of an immunogenic composition, comprising:
a) sequencing the circulating tumor DNA from a biological sample of a subject to generate circulating tumor DNA sequence data; and
b) analyzing the circulating tumor DNA sequence data to generate a numerical probability score of the prevalence of one or more tumor-specific neoantigens in the circulating tumor DNA;
wherein a higher numerical probability score relative to a low numerical probability score indicates that the one or more tumor-specific neoantigens is present in the circulating tumor DNA and that the one or more tumor-specific neoantigens has a lower prevalence in a tumor of the subject relative to before administration of the immunogenic composition; and
wherein a lower numerical probability score relative to a higher numerical probability score indicates that the one or more tumor specific neoantigens is present in a low amount or not present in the circulating tumor DNA and that the one or more tumor-specific neoantigens has a higher prevalence in the tumor of the subject relative to before administration of the immunogenic composition;
thereby evaluating the efficacy of the immunogenic composition.

2. A method for treating cancer, comprising:
a) administering to a subject in need thereof a first immunogenic composition;
b) sequencing circulating tumor DNA from a biological sample from a subject to generate circulating tumor DNA sequence data; and
c) analyzing the circulating tumor DNA sequence data to generate a numerical probability score of the prevalence of one or more tumor-specific neoantigens in the circulating tumor DNA;
wherein a higher numerical probability score indicates that the one or more tumor-specific neoantigens is present in the circulating tumor DNA and that the one or more tumor-specific neoantigens has a lower prevalence in a tumor of the subject relative to before administration of the first immunogenic composition; and
wherein a lower numerical probability score indicates that the one or more tumor specific neoantigens is present in a low amount or not present in the circulating tumor DNA and that the one or more tumor-specific neoantigens has a higher prevalence in the tumor of the subject relative to before administration of the first immunogenic composition;
d) generating a second immunogenic composition.

3. The method of claim 1, further comprising generating a second immunogenic composition.

4. The method of claim 1, wherein the subject was previously administered or will be prospectively administered an immunogenic composition.

5. The method of claim 1, wherein the immunogenic composition is a tumor-specific neoantigen immunogenic composition or a tumor-associated antigen-based immunogenic composition.

6. The method of claim 2, wherein the first immunogenic composition and the second immunogenic composition are tumor-specific neoantigen immunogenic compositions or tumor-associated antigen-based immunogenic compositions.

7. The method of claim 2, wherein the second immunogenic composition comprises one or more tumor-specific neoantigens that have a lower numerical probability score relative to a higher numerical probability score.

8. The method of claim 2, wherein the second immunogenic composition comprises one or more tumor-specific neoantigens not present in the circulating tumor DNA.

9. The method of claim 2, wherein the second immunogenic composition comprises one or more tumor-specific neoantigens that are present in the circulating tumor DNA.

10-15. (canceled)

16. The method of claim 1, wherein the circulating tumor DNA is obtained from a blood sample, a serum sample, a plasma sample, a lymphatic fluid sample, a urine sample, or a cerebrospinal fluid sample.

17-22. (canceled)

23. A method for determining whether a subject has cancer relapse, comprising:
a) sequencing circulating tumor DNA from a biological sample of the subject to generate circulating tumor DNA sequence data; and
b) analyzing the circulating tumor DNA sequence data to generate a numerical probability score of the prevalence of one or more tumor-specific neoantigens in the circulating tumor DNA;
wherein a higher numerical probability score relative to a low numerical probability score indicates that the one or more tumor-specific neoantigens is present in the circulating tumor DNA and the subject has cancer relapse; and
wherein a lower numerical probability score relative to a higher numerical probability score indicates that the one or more tumor specific antigens is present in a low amount or not present in the circulating tumor DNA and that the subject does not have cancer re-lapse;
thereby determining whether the subject has cancer relapse.

24. The method of claim 23, further comprising generating an immunogenic composition.

25. The method of claim 23, wherein the subject was previously administered or will be prospectively administered an immunogenic composition.

26. The method of claim 24, wherein the immunogenic composition is a tumor-specific neoantigen immunogenic composition or a tumor-associated antigen-based immunogenic composition.

27. The method of claim 24, wherein the immunogenic composition comprises one or more tumor-specific neoantigens that have a higher numerical probability score relative to a lower numerical probability score.

28. The method of claim 24, wherein the immunogenic composition comprises one or more tumor-specific neoantigens present in the circulating tumor DNA.

29. The method of claim 23, further comprising clustering the one or more tumor-specific neoantigens identified in the circulating tumor DNA to identify one or more tumor-subclones.

30. (canceled)

31. The method of claim 29, wherein the tumor-specific neoantigens identified in the circulating tumor DNA and tumor-specific neoantigens identified as being associated with one or more tumor-subclones of the tumor-specific neoantigens identified in the circulating tumor DNA are included in the immunogenic composition.

32. (canceled)

33. The method of claim 23, wherein the circulating tumor DNA is obtained from a blood sample, a serum sample, a plasma sample, a lymphatic fluid sample, a urine sample, or a cerebrospinal fluid sample.

34-40. (canceled)

41. The method of claim 2, wherein
the circulating tumor DNA is obtained from tumor draining veins (e.g., immediately downstream or locoregional).

42. The method of claim 2, wherein the circulating tumor DNA is obtained from lymph downstream (e.g., immediately downstream) from a tumor.
